(19) United States
(12) Patent Application Publication
(10) Pub. No.: US 2022/0095998 A1
(43) Pub. Date: Mar. 31, 2022

(54) HYPERSPECTRAL IMAGING IN AUTOMATED DIGITAL DERMOSCOPY SCREENING FOR MELANOMA

(71) Applicant: THE ROCKEFELLER UNIVERSITY, New York, NY (US)

(72) Inventor: Daniel Gareau, New York, NY (US)

(73) Assignee: THE ROCKEFELLER UNIVERSITY, New York, NY (US)

(21) Appl. No.: 17/369,551

(22) Filed: Jul. 7, 2021

Related U.S. Application Data
(63) Continuation of application No. PCT/US2020/012724, filed on Jan. 8, 2020.
(60) Provisional application No. 62/789,652, filed on Jan. 8, 2019.

(51) Int. Cl. A61B 5/00; G06T 7/00

(52) U.S. Cl. CPC A61B 5/444; A61B 5/0075; A61B 5/7267; A61B 5/7253; G06T 2207/20084; G06T 2207/30088; G06T 2207/30096; G06T 2207/10036; G06T 2207/20081; G06T 7/0012

(57) ABSTRACT

Hyperspectral dermoscopy images obtained in N wavelengths in the 350 nm to 950 nm range with a hyperspectral imaging camera are processed to obtain imaging biomarkers having a spectral dependence. Machine learning is applied to the imaging biomarkers to generate a diagnostic classification.

### CROSS REFERENCE TO RELATED APPLICATIONS

This application is a continuation of PCT International Application No. PCT/US2020/012724, International Filing Date Jan. 8, 2020, claiming the benefit of U.S. Provisional Application Ser. No. 62/789,652, filed on Jan. 8, 2019 and entitled HYPERSPECTRAL IMAGING IN AUTOMATED DIGITAL DERMOSCOPY SCREENING FOR MELANOMA, which is incorporated in its entirety herein by reference.

### BACKGROUND OF THE INVENTION

Early melanoma detection decreases morbidity and mortality. Early detection classically involves dermoscopy to identify suspicious lesions for which biopsy is indicated. Biopsy and histological examination then diagnose benign nevi, atypical nevi, or cancerous growths. With current methods, a considerable number of unnecessary biopsies are performed because only a fraction of all biopsied, suspicious lesions are actually melanomas. Thus, there is a need for more advanced noninvasive diagnostics to guide the decision to biopsy.

The detection of melanoma clinically can be visually challenging and often relies on the identification of hallmark features including asymmetry, irregular borders, and color variegation to identify potentially cancerous lesions. In clinical practice, only 11% of all biopsied, suspicious lesions are melanomas and thus a substantial number of unnecessary biopsies are performed. The dermatoscope aids in detection of melanoma by providing magnified and illuminated images. However, even among expert dermoscopists, the sensitivity of detecting small melanomas (<6 mm) is as low as 39%. Thus, despite evidence that early detection decreases mortality, considerable uncertainty surrounds the effectiveness of state of-the-art technology in routine melanoma screening.

In this context, clinical melanoma screening is a signal-detection problem, which guides the binary decision for or against biopsy. Physicians screening for melanoma prior to the (gold standard) biopsy may be aided or, in some cases, outperformed by artificial-intelligence analysis. However, deep-learning dermatology algorithms cannot show a physician how a decision was arrived at, diminishing enthusiasm in the medical community. Thus, as regards melanoma detection, there is an unmet need for clinically interpretable machine vision and machine learning to provide transparent assistance in medical diagnostics. Improved clinical screening may prevent some of the roughly 10,000 annual deaths from melanoma in the United States.

Dermoscopy, in which a liquid interface or cross-polarizing light filters allow visualization of subsurface features, including deeper pigment and vascular structures, has been shown to be superior to examination with the naked eye; however, it remains limited by significant inter-physician variability and diagnostic accuracy is highly dependent on user experience. Studies using test photographs and retrospective analyses report increased diagnostic accuracy with the addition of dermoscopy criteria. In one study, dermatologists with at least 5 years of experience using dermoscopy showed a 92% sensitivity and 99% specificity in detecting melanoma, but this dropped to 69% and 94% with inexperienced dermatologists (less than 5 years of experience), respectively. Even more concerning, the use of dermoscopy by inexperienced dermatologists may result in poorer performance compared to examination with the naked eye.

The first computer-aided diagnosis system for the detection of melanoma was described in 1987. Since then, a variety of non-invasive in vivo imaging methodologies have been developed, including digital dermoscopy image analysis (DDA), total body photography, laser-based devices, smart phone-based applications, ultrasound, and magnetic resonance imaging. The primary challenge with clinical application of these technologies is obtaining a near perfect sensitivity, as a false negative, or Type II melanoma screening error, can have a potentially fatal outcome.

Current hyperspectral/multispectral imaging methods on the market include MelaFind® and SIAScope®. Mela-Find® (MELA Sciences, Irvington, N.Y.) is a handheld device that images from 430 nm (blue) to 950 nm (near infrared), and was FDA approved in 2011 for melanoma detection. Results from a multi-center prospective trial in 2011 reported a sensitivity of 98.2% and specificity of 9.5%. In a follow-up study using a test set of 47 lesions to compare MelaFind performance to that of dermatologists in detecting melanoma, the authors report a sensitivity of 96% and specificity of 0.08%. MelaFind recommended biopsy in 44 lesions and no biopsy in 3. In the three lesions that were not biopsied, one was diagnosed as melanoma. In a study of 160 board-certified dermatologists who were asked to evaluate 50 randomly ordered pigmented lesions, the sensitivity and specificity for diagnosing melanoma significantly increased after physicians were provided MelaFind analysis of lesions from 76% to 92% and from 52% to 79%, respectively. However, there is still significant debate as to whether MelaFind is a useful tool to guide dermatologists, the concern being that the device almost always recommends biopsy.

Spectrophotometric Intracutaneous Analysis (SIAscopy™, Astron Clinica, UK) was first introduced in 2002 as an imaging technology that produces spectrally filtered images in the visible and infrared spectra (400-1000 nm). The first clinical trial with SIAscopy demonstrated a sensitivity of 82.7% and specificity of 80.1% for melanoma in a dataset of 348 pigmented lesions (52 melanomas). However, when implemented in a melanoma screening clinic, the SIAscope did not improve the diagnostic abilities of dermatologists. Further studies demonstrated poor correlation between SIAscopy analysis and histopathology in both melanoma and nonmelanoma lesions and worse accuracy than dermoscopy. Of note, direct comparison of devices to other systems on the market is limited, as diagnostic performance of a device varies with the difficulty of lesions included in analysis, as well as the proportion of atypical nevi in the benign set.

DDA systems have attempted to decrease inter-physician variability and standardize dermoscopic analysis by incorporating quantitative parameters such as colorimetric and geometric evaluation. There are a variety of proprietary DDA instruments on the market, although none have yet demonstrated a reproducibly high sensitivity and specificity for melanoma detection. SolarScan® (Polartechnics Ltd, Sydney, Australia), for example, is an automated digital dermoscopy instrument that extracts lesion characteristics from digital images and then compares them to a database of benign and malignant lesions. In clinical studies, SolarScan® demonstrated a sensitivity of 91% and specificity of 68% for detecting melanoma. In the evaluation of another DDA, the FotoFinder Mole-Analyzer®, a 15-year retrospective study evaluated diagnostic performance in 1,076 pigmented skin lesions, reporting a low diagnostic accuracy with a sensitivity of 56% and specificity of 74%, which was significantly lower than previous reports with the same system. There remains a wide variation in sensitivity and specificity amongst the current DDA systems, ranging from 56% to 100% and 60% to 100%, respectively. Further investigation into the diagnostic accuracy of these DDA systems is needed for more standardized, reproducible results.

Deep learning, automated processing diagnostic devices are rapidly transforming healthcare due to their remarkable predictive power, yet careful considerations of biases in training data present ethical concerns and limit adoption of artificial intelligence. Black box deep learning approaches such as convolutional neural networks (CNN) may be inappropriate for stand-alone diagnostic medical decision-making because these algorithms cannot be held liable for screening errors and neither can physicians who use them without understanding the underlying computational diagnostic mechanics.

### SUMMARY OF THE INVENTION

The present invention represents an advance in computer-aided dermoscopic screening for melanoma, in embodiments achieving near perfect sensitivity, and specificity of 36% in diagnosis.

A digital melanoma “imaging biomarker” is a quantitative metric extracted from a dermoscopy image by one or more computer algorithms that is high for melanoma (1) and low for a nevus (0). These imaging biomarkers measure features that are associated with pathological and normal features which are thereafter input to more complex machine learning algorithm(s) to create diagnostic classifiers. Thus, “imaging biomarker” is defined as a quantitative feature extracted from one or more images that is higher for melanoma than for a nevus. Examples of melanoma imaging biomarkers include symmetry, border, brightness, number of colors, organization of pigmented network pattern, and others, as described below.

According to embodiments of the invention, screening algorithms are generated with the aid of artificial intelligence from a set of imaging biomarkers to transform the set of imaging biomarkers into a risk score that can be used to classify a lesion as a melanoma or a nevus by comparing the score to a classification threshold. Melanoma imaging biomarkers have been shown to be spectrally dependent in the hyperspectral range, beyond the standard Red, Green, Blue (RGB) color channels, and hyperspectral imaging further enhances diagnostic power by leveraging this spectral dependence.

“Spectral” imaging is imaging obtained in red green and blue (RGB) color channels, “multispectral” imaging utilizes more than 3 up to about 10 wavelengths or “color channels” and “hyperspectral” means that more than 10 separate color channels are used to obtain and process images.

In one aspect the invention is directed to the use of imaging biomarkers described in U.S. Pat. Nos. 10,182,757 and 10,307,098, and U.S. Patent Application Publication No. 2018/0235534 (all of which are by the inventor herein and are incorporated by reference) over a hyperspectral range of wavelengths, and supplying the spectral biomarker information to algorithms, including machine learning algorithms to obtain enhanced detection of skin disease, such as melanoma.

U.S. Patent Application Publication No. 2018/0235534 described two types of imaging biomarkers: single color channel imaging biomarkers derived from gray scale images extracted from individual color channels (Red, Green, Blue (RGB)), and multicolor imaging biomarkers that were derived from all color channels simultaneously. An example of a multi-color imaging biomarker would be the number of dermoscopic colors contained in the lesion, since the definition of a color includes relative levels of intensity for the red, green, and blue channels. These melanoma imaging biomarkers are spectrally dependent in RGB color channels, with most imaging biomarkers showing statistical significance for melanoma detection in the red or blue color channels. According to embodiments of the invention, the spectral dependence of the imaging biomarkers over the entire hyperspectral spectrum is leveraged to improve diagnostic accuracy using the same melanoma imaging biomarkers over a wide range of wavelengths (350 nm-950 nm) in combination with machine learning algorithms to result in enhanced melanoma detection. Spectral fitting using the hyperspectral wavelengths allows modelling a second type of biomarker, which has a single value obtained from the full hyperspectral range. Examples of the second type of biomarker include blood volume fraction (BVF) and oxygen saturation (O₂-Sat).

According to one aspect of the invention, an ensemble classifier (“Eclass”) composed of “non-deep” machine learning algorithms providing a set of imaging biomarkers that quantify medically relevant features may be more accountable and more accurate than simply unleashing CNN on the raw images to choose salient features freely. Machine learning-based digital diagnosis for earlier detection is potentially valuable, particularly for high-risk, fast-growing melanomas where a 6-month diagnosis delay may allow growth of a melanoma from 0.052 to 0.120 mm in Breslow thickness, a metastasis risk, and pre-existing theoretical frameworks (e.g. dermoscopy) offer more appropriate machine learning applications in medical imaging because they can translate between machine intelligence and human intelligence.

Thus, in one aspect, the invention is a method of dermoscopic screening of a lesion, comprising the steps of: imaging a lesion on a subject's skin under a set of N illumination spectra to obtain a sequenced set of N images, each said image comprised of image data; wherein the set of N illumination spectra is hyperspectral; calculating at least one of a first type of biomarker and a second type of biomarker, wherein the first type of biomarker comprises M imaging biomarker values and is calculated by transforming said image data of said N images into said M imaging biomarker values; wherein the first type of imaging biomarker value varies as a function of the N illumination spectra; and wherein the second type of biomarker is calculated from all of said N illumination spectra at each pixel, so that said second type of biomarker has only one value for said N illumination spectra at each pixel; and applying a trained transformation algorithm to transform at least one of the first type of biomarker and the second type of biomarker into a classification indicating the likelihood that the lesion is a skin disease.

In embodiments, both the first type of biomarker and the second type of biomarker are calculated, and the trained transformation algorithm is applied to both the first and second types to obtain said classification.

In embodiments, the trained transformation algorithm comprises one, some or preferably all of the following non-deep learning algorithms applied to said first and/or second type biomarkers to obtain said classification: (1) logistic regression; (2) feed-forward neural networks with a single hidden layer; (3) linear and support vector machines radial (SVM); (4) decision tree algorithm for classification problems; (5) Random Forests; (6) linear discriminant analysis (LDA); (7) K-nearest neighbors algorithm (KNN); and (8) Naive Bayes algorithm. As described below, these “non-deep” machine learning “Eclass” transformation algorithms may be used instead of deep learning algorithms such as convolutional neural network (CNN). As broadly understood, however, “transformation algorithm” may include both types, Eclass and “deep learning” algorithms.

In embodiments of the invention, a second type of biomarker includes blood volume fraction (BVF) and oxygen saturation (O₂-Sat) to evaluate the metabolic state of tissue in the lesion.

In embodiments, a center frequency of a first spectrum of said set of N illumination spectra is separated from a center frequency of an adjacent second spectrum by approximately a half-power bandwidth of said first spectrum, such that when the N illumination spectra are normalized to have an area of unity, the first spectrum and the second spectrum intersect at respective half-power points, as described below.

In embodiments, the spectra of the individual LEDs in the system may be selected by selecting each of the N illumination spectra by dividing an entire wavelength range of said spectra into wavelength segments each approximately equal to a half-power bandwidth of one of said illumination spectra, and using an illumination source emitting at a wavelength in said segment.

In another aspect, embodiments of the invention include an apparatus for imaging and analysis of a lesion on a subject's skin, comprising: an illumination system controlled by a processor to sequentially illuminate a lesion on a subject's skin with N illumination spectra; a camera controlled by a processor to obtain a sequenced set of N images of said lesion in said N illumination spectra. A processor (which may be in a housing onboard the camera) is adapted to transform image data of said N images into M imaging biomarker values and a second processor (which may be remote from the camera) is adapted to apply a trained transformation algorithm to transform said M imaging biomarker values into a classification indicating the likelihood that the lesion is skin disease, such as melanoma

In embodiments, the illumination system comprises a set of LEDs for each of said N illumination spectra, said LEDs emitting wavelengths in a range of 350 nm to 950 nm. The LEDs may be arranged such that a center frequency of a first spectrum of said set of N illumination spectra is separated from a center frequency of an adjacent second spectrum by approximately a half-power bandwidth of said first spectrum, such that when the N illumination spectra are normalized to have an area of unity, the first spectrum and the second spectrum intersect at respective half-power points.

In embodiments, the apparatus comprises a housing, wherein the housing attaches, in a self-contained unit, a transparent flat surface to position against a lesion to define a distal imaging plane, a lens, a camera, a motor, gearing; and a camera processor controlling the camera and the motor to obtain said N images. The housing may also include, in the same self-contained unit, a first processor adapted to transform the N sequenced images into M biomarkers data and encrypt and transmit said M biomarkers data. Alternatively, or complementarily, the first processor may be configured to obtain a second type of biomarker calculated from all of said N illumination spectra at each pixel, so that said second type of biomarker has only one value for said N illumination spectra at each pixel.

In embodiments, the apparatus housing further comprises an imaging window and a space adapted to securely receive a mobile phone adapted to display an in-line view of the lesion on a display of the smart phone, and wherein the apparatus further comprises an app to connect the mobile phone to the camera processor to create a secondary display.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 depicts the value of two imaging biomarkers obtained from a single lesion as a function of wavelength;

FIG. 2A depicts the spacing and overlap of 21 hyperspectral color channels according to one embodiment of the invention, ranging from the ultraviolet (UV)A (350 nm) to the near infrared (IR) (950 nm) used in a method according to one embodiment of the invention;

FIG. 2B schematically depicts components of an imaging and dermoscopic analysis apparatus according to the invention;

FIG. 3A is an RGB image of a lesion according to an embodiment of the invention with a pixel identified;

FIG. 3B depicts a blood volume fraction map produced by fitting the spectrum at each pixel according to embodiments of the invention;

FIG. 3C depicts an oxygen saturation map produced by fitting the spectrum at each pixel according to embodiments of the invention;

FIG. 3D depicts a melanin factor map produced by fitting the spectrum at each pixel according to embodiments of the invention;

FIG. 3E depicts an example of hyperspectral fitting of a single pixel in the image of FIG. 3A for mapping of blood volume fraction, oxygen saturation and melanin as shown in FIG. 3B, FIG. 3C and FIG. 3D;

FIG. 4 is a receiver operator characteristic (ROC) curve for melanoma detection in hyperspectral images;

FIG. 5 is a schematic flow chart showing a sequence for obtaining, hyperspectral images, imaging biomarkers and diagnostic classifiers according to embodiments of the invention; and

FIG. 6A shows ROC curves comparing Eclass “non-deep” learning and CNN deep learning approaches to automated screening and FIG. 6B shows an example of imaging biomarkers that may be fed to the Eclass non deep machine learning algorithms.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

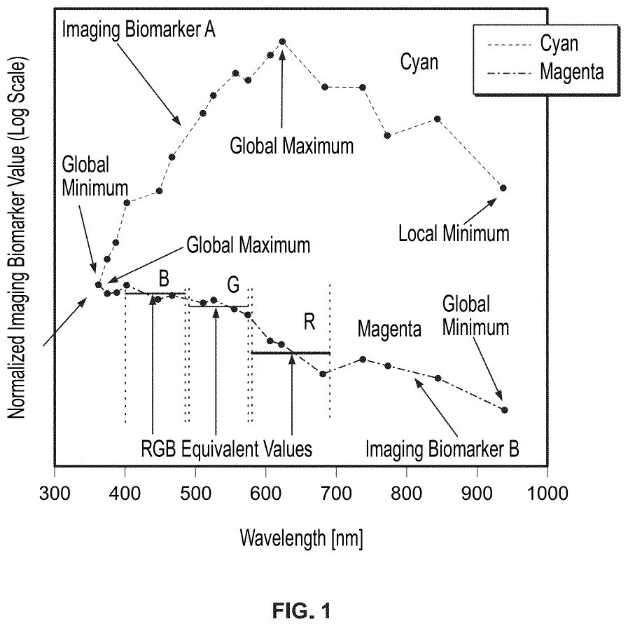

FIG. 1 shows the spectral dependence of two imaging biomarkers on one sample lesion over the entire spectrum, as a function of wavelength, providing evidence that a machine learning algorithm utilizing a range of wavelengths may achieve higher sensitivity and specificity compared to RGB equivalent values. The two imaging biomarkers selected for analysis were the most melanoma-predictive RGB biomarkers identified in the aforesaid U.S. Patent Application Publication No. 2018/0235534 (i.e., “optimum imaging biomarkers”).

The optimum imaging biomarker value for imaging biomarker A (cyan) would be the lowest value (global minimum), which would be in the ultraviolet. Meanwhile, the global minimum of imaging biomarker B (magenta) would be in the infrared. In the case of a melanoma, the optimum imaging biomarker value for imaging biomarker A (cyan) would be the highest value (global maximum), which would be in the red color channel Meanwhile, the global maximum of imaging biomarker B (magenta) would be in the ultraviolet. Thus, the optimum imaging biomarker values in these examples would not be captured with RGB imaging alone. Further, diagnostic utility may be derived from image heterogeneity measures in the ultraviolet range since ultraviolet light interacts with superficial cytological and morphological atypia, targeting superficial spreading melanoma.

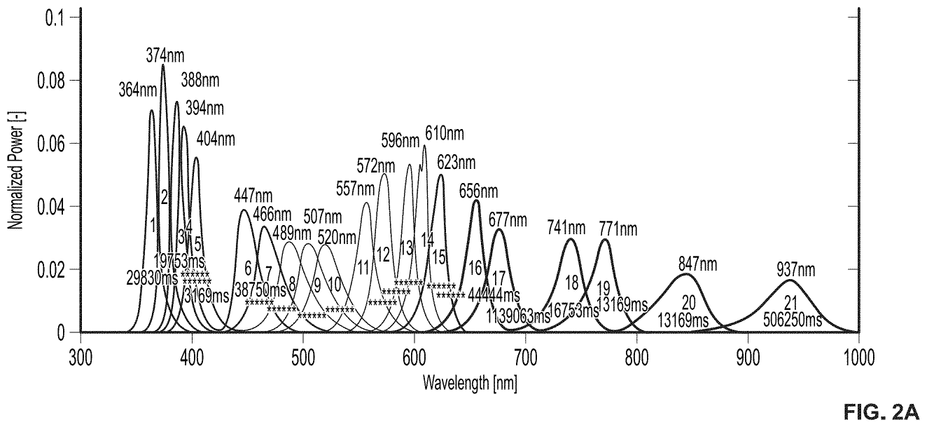

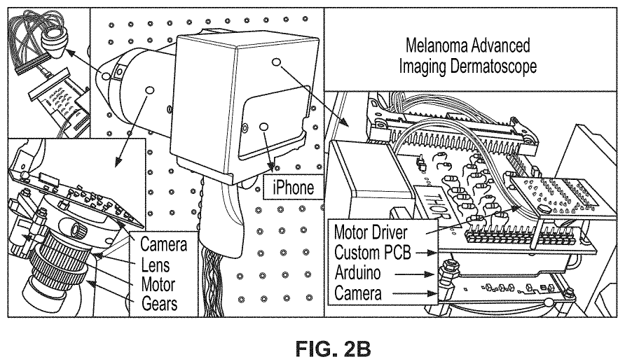

FIG. 2B schematically depicts an embodiment of the apparatus, also referred to herein as the melanoma Advanced Imaging Dermatoscope (mAID). The mAID is a non-polarized light-emitting diode (LED)-driven hyperspectral camera including lens, motor and gearing adapted to sequentially illuminate the skin with 21 different wavelengths of light ranging from the ultraviolet (UV)A (350 nm) to the near infrared (IR) (950 nm) (FIG. 2A), which is referred to as the range of the N illumination spectra. This example is not to be deemed as limiting the invention, which may use a different number N of hyperspectral wavelengths and may employ an illumination source other than an LED. In the embodiment shown, each LED is associated with a spectral curve, as shown in FIG. 2A Images are collected using a high sensitivity gray scale charge-coupled device (CCD) array (Mightex Inc., Toronto, Ontario, CA). A transparent flat surface, such as glass, is provided at the front end of the device to position against a lesion to define a distal imaging plane, similar to a dermatoscope. In comparison to a standard digital camera, which captures light at three relatively broad wavelength bands of light (RGB), the mAID device achieves about five times better spectral resolution as well as widened spectral range.

The LEDs were chosen such that the spectrum of each LED is separated from its spectral neighbor by a spectral distance that is approximately the full-width at half-maximum of the LED spectrum. This scenario leads to LED spectra that, when normalized to have an area of unity, overlap at the half maximum point. Thus, as shown in FIG. 2A, a center frequency of a first spectrum of said set of N illumination spectra is separated from a center frequency of a second spectrum by approximately a half-power bandwidth of said first spectrum, such that when the N illumination spectra are normalized to have an area of unity, the first spectrum and the second spectrum intersect at respective half-power points. This approach approximates Nyquist sampling and results in an appropriate number of LEDs so as to not over-sample spectrally. There are between one and eight LEDs per wavelength: four for UV wavelength, eight for IR wavelength, and one for most of the visible wavelengths. The number of LEDs per wavelength was empirically determined by evaluating image brightness. The image sensor may be less sensitive to the non-visible spectra and brightness/intensity of the LED illumination may be increased accordingly. As used herein, the term “LED” may refer to one LED or multiple LEDs if more than one LED is used to obtain more intensity at a given wavelength. A person of ordinary skill in the art recognizes that the specified spectral distance is “approximate”, in the sense that the spacing may be varied slightly to accommodate commercially available LEDs and different performance among LEDs or in view of other engineering considerations.

Of note, there is no fluorescence as there is no filter to block the reflected UVA light, which is stronger than the fluorescent emission. It is possible that there is unwanted fluorescence, but it is small compared to the reflectance signal, and therefore negligible. There is no photobleaching as the irradiance incident on the skin is several fold less than sunlight and one second of sunlight does not cause photobleaching.

Other notable features of the device include a 28 mm imaging window and a mobile phone embedded in its back surface to display a live, “in-line” view of the target skin lesion. The mobile phone is not used for processing, but is connected to the device via the TwoMon app (DEVGURU Co. Ltd, Seoul, South Korea) to create a secondary display to help align the device properly with the target lesion.

In terms of safety, the total light dose is less than one second of direct sunlight exposure and the mAID holds an abbreviated investigational device exemption from the FDA.

The protocol for imaging with the mAID device includes placing the imaging head directly onto the skin after applying a drop of immersion media such as hand sanitizer. After automated focusing, the device sequentially illuminates the skin with 21 different wavelengths of light.

The operator needs to be properly trained in use of the mAID. Movement during imaging can lead to a series of laterally sliding positions on the skin, and hence the lesion and its diagnostic morphology will not be spatially coherent. In addition, the presence of hair and bubbles in the imaging medium can interfere with image analysis. This presents a challenge as many of the lesions dermatologists evaluate are in hair bearing regions.

Discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, on board or remote from the camera, that manipulates and/or transforms data represented as physical (e.g., electronic) quantities within the computer's registers and/or memories into other data similarly represented as physical quantities within the computer's registers and/or memories or other non-transitory information storage medium that may store instructions to perform operations and/or processes. As used herein, the terms “controller” and “controls” likewise may refer to a computer onboard the camera or in a remote location.

The processing functions may be shared between first and second processors. The first processor is typically an onboard processor such as circuit board adapted to drive the camera and illumination system to acquire the image data and provide real time information display to the user. The first processor may transmit image data to a second processor adapted to perform data-intensive processing functions which cannot readily be provided as real time display. The second processor may deliver messages back to the first processor for display. The second processor, if present, is typically a remote processor. The second processor may create data files, image files, and the like, for later use. In embodiments the first and second processor are attached in a housing. The designations “processor”, “first processor”, “second processor”, and “camera processor” are for convenience only based on the functions being performed. Each said processor may be comprised of multiple components or more than one processor may be integrated in a single component.

The entire process takes less than four minutes including set up and positioning, with the collection of images requiring 20 seconds. In addition, there is no discomfort for the patient. The mAID device may include a processor adapted to automatically encrypt and transfer hyperspectral images from the clinical site of imaging to the site of analysis over a secure internet connection.

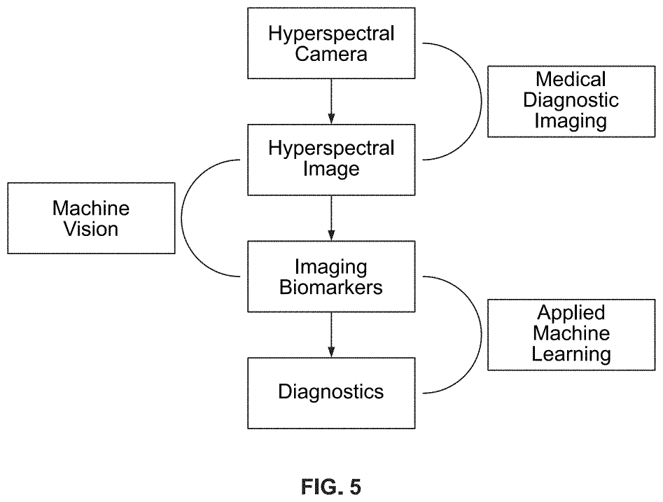

FIG. 5 schematically depicts an overall process flow, in which medical diagnostic imaging 51 refers to obtaining the hyperspectral images, substantially as disclosed in the prior patents incorporated by reference. Machine vision 52 refers to obtaining first and second type imaging biomarkers from the hyperspectral images, which is a task of the “first processor” which is typically (but not necessarily) onboard the imaging device. Applied machine learning 53 refers to a transformation algorithms, one or both Eclass type or “deep learning”, as discussed below which is a task of the “second processor” which is typically (but not necessarily) remote from the imaging device.

A clinical study was approved by the University of California, Irvine Institutional Review Board. After obtaining informed consent, 100 pigmented lesions from 91 adults 18 years and over who presented to the Department of Dermatology at the University of California, Irvine from December 2015 to July 2018 underwent imaging with the mAID hyperspectral dermatoscope prior to removal and histopathological analysis. All imaged lesions were assessed by dermatologists as suspicious pigmented lesions requiring a biopsy. After obtaining the final histopathologic diagnoses, 30 lesions were excluded from analysis due to their non-binary classification (i.e., not a melanoma or nevus). These categories included atypical squamous proliferation (1), basal cell carcinoma (9), granulomatous reaction to tattoo pigment (1), lentigo (4), lichenoid keratosis (1), melanotic macule (1), seborrheic keratosis (9), splinter (1), squamous cell carcinoma (2), and thrombosed hemangioma (1). Seventy mAID hyperspectral images then underwent automated computer analysis to create a set of melanoma imaging biomarkers. These melanoma imaging biomarkers were derived using hand-coded feature extraction in the Matlab programming environment Images from 52 of the total 70 pigmented lesions were successfully processed. The remaining 18 images were excluded due to one or more of the following errors in processing: bubbles in the imaging medium, image not in focus, camera slipped during imaging, or excessive hair was present in the image obscuring the lesion. In the application of machine learning algorithms, ground truth was the histopathological diagnosis of melanoma or nevus that was accessed automatically during learning. The machine learning, with the melanoma imaging biomarkers as inputs, was trained to output a risk score which was the likelihood of a melanoma diagnosis. In this way, the machine learning created the best transformation algorithm to arrive at the result of the invasive test but using only the noninvasive images acquired prior to the biopsy. A summary of the melanoma classification algorithms used is listed in Table 1. As would be recognized by a person having ordinary skill in the art, these are “non-deep” learning algorithms and in embodiments, one, some or preferably all of said classification algorithms are applied to the imaging biomarkers to obtain a classification which may be shown to be more accurate than a deep learning algorithm.

TABLE 1
Method Description

| | |
|---|---|
| LoR | Logistic regression within the framework |
| | of Generalized Linear Models |
| NN | Feed-forward neural networks with a |
| | single hidden layer |
| SVM | (linear and Support vector machines radial) |
| DT | C5.0 decision tree algorithm for |
| | classification problems |
| RF | Random Forests |
| LDA | Linear discriminant analysis |
| KNN | K-nearest neighbors algorithm developed |
| | for classification |
| NB | Naive Bayes algorithm |

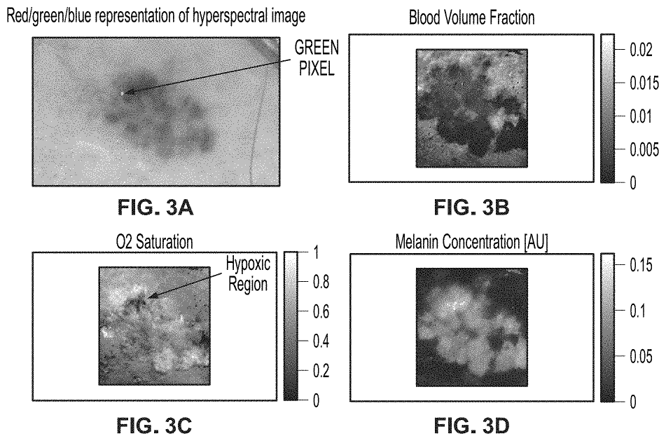

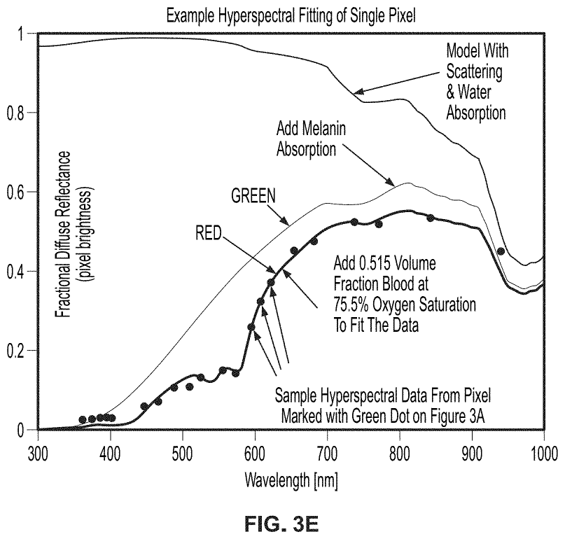

The derivation of melanoma imaging biomarkers and corresponding methods of image analysis have been previously described in the aforesaid U.S. patents incorporated by reference. The extension of single color channel imaging biomarkers to hyperspectral imaging entailed calculating (in this case) 21 values for each imaging biomarker per hyperspectral image—one for each of the 21 color channels in the hyperspectral image. Using these quantitative metrics, the algorithm generated an overall Q-score for each image—a value between zero and one in which a higher number indicates a higher probability of a lesion being cancerous Images were also processed by spectral fitting to produce blood volume fraction (BVF) and oxygen saturation (O₂-Sat), which are candidate components in identifying metabolic and immune irregularity in melanomas (FIG. 3). These imaging biomarkers obtained by spectral fitting are a second type of biomarker. Thus the M imaging biomarkers are of two classes: a second type of imaging biomarkers where each imaging biomarkers is computed using the entire spectrum and a first type of imaging biomarkers where each biomarker is computed using a single wavelength at a time and each imaging biomarker of this type comes in N (in this case 21) values, whereas biomarkers of the second type come in only one value (calculated using all the illumination spectral values N).

Spectral light transport in turbid biological tissues is a complex phenomenon that gives rise to a wide array of image colors and textures inside and outside the visible spectrum. To understand the degree to which different wavelengths interact with tissue at different depths in the skin, a Monte Carlo photon transport simulation was adapted to run at all the hyperspectral wavelengths. The simulation modeled light transport into and out of pigmented skin lesions. Modeling involved two steps: (i) 20 histologic sections of pigmented lesions stained with Melan-A were imaged with a standard light microscope to become the model input; (ii) light transport at 40 wavelengths in the 350-950 nm range was simulated into and out of each input model morphology. First, a digital image of the histology was automatically segmented into epidermal and dermal regions using image processing. Each region was assigned optical properties appropriate for each tissue compartment (i.e., the epidermis had high absorption due to blood and the dermis had an absorption spectrum dominated by hemoglobin but also some melanin) The escaping photons were scored by simply checking, at each propagation step, if they had crossed the boundary of the surface of the skin (all other boundaries were handled with a matched boundary condition). For escaping photons, the numerical aperture of the camera was transformed into a critical angle. If the photons escaped at an angle that was inside the critical angle, their weight at time of escape was added to the simulated pixel brightness at that image point. The positions and directions were scored for each escaping photon as well as the maximum depth of its penetration.

To generate BVF and O₂-Sat which are depicted in FIG. 3B, FIG. 3C and FIG. 3E a tissue phantom was constructed composed of scattering collagen, keratin, and melanin. The predicted spectrum is shown in FIG. 3 (black), obtained using diffusion theory modified for simulating diffuse reflectance of skin lesions. The spectrum from each pixel was assumed to follow the well-established diffusion theory of photon transport. However, as a consequence of illuminating and detecting from the entire field, illumination occurs both far from detection and on top of the detection points. In the dermis, the absorption coefficient is assumed to be homogenous and contributed to by a fraction of water times the absorption coefficient of water, a fraction of deoxyhemoglobin times the absorption of deoxyhemoglobin, a fraction of oxyhemoglobin times the absorption of oxyhemoglobin. Melanin was modeled in the dermis the same as was the previously mentioned chromophores but with a proportional “extra melanin” factor acting as a transmission filter in the superficial epidermis. This last feature is a departure from simple diffusion theory and it models the dermis as source of diffuse reflectance that transmits through the epidermis, where an extra amount of melanin that is proportional to the dermal melanin (to maintain only one fitting parameter for melanin concentration) attenuates the diffuse reflectance escaping the tissue.

Monte Carlo simulation showed that the mean penetration depth of escaping light was a thousand-fold greater than its wavelength. For example, 350 nm light penetrated 350 mm into the tissue, 950 nm light penetrated 950 mm into the tissue and the relationship was linear at the 40 wavelengths between these two points.

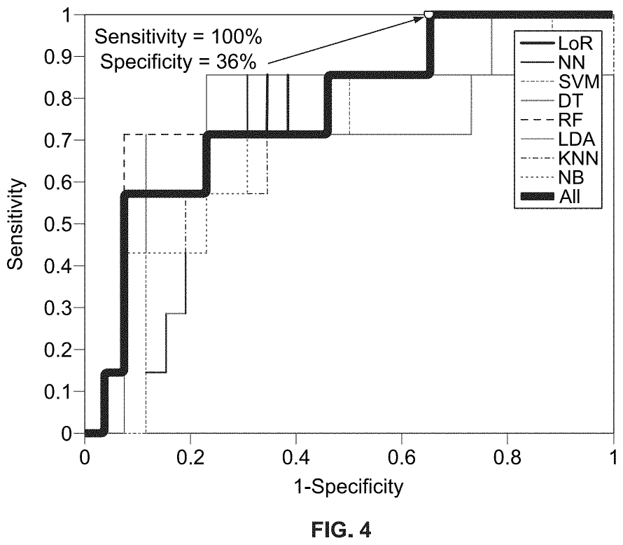

Of the 52 pigmented lesions that were successfully processed with hyperspectral imaging, 13 (25%) were histologically diagnosed as melanoma and 39 (75%) were diagnosed as nevi. Sensitivity, specificity, and diagnostic accuracy were calculated from the ROC curves (FIG. 4).

The corresponding confusion matrix is shown in Table 2. “Specificity” refers to the tendency to avoid a false positive diagnosis, which must be increased to avoid unnecessary and costly biopsies, while “sensitivity” refers to the tendency to avoid a false negative, which must approach perfection to avoid a potentially fatal misdiagnosis. These statistically related quantities are inevitably in tension. Table 2 shows the results of the FIG. 4 displayed in a “Confusion Matrix Table”, correlating the 100% Sensitivity and 36% specificity achieved according to the invention. According to embodiments of the invention, sensitivity of the apparatus and method for detecting melanoma approaches 100%, meaning that the likelihood of a false negative diagnosis is exceedingly rare. In embodiments, sensitivity “approaching 100%” means greater than 99.5% sensitivity, in another embodiment, sensitivity “approaching 100%” may be statistically indistinguishable from 100%. In any event, these results may reflect a given statistical sample and are provided as a benchmark.

TABLE 2

| N = 52 | Negative | Positive | |
|---|---|---|---|
| No disease | TN = 14 | FP = 25 | 39 |
| Disease | FN = 0 | TP = 13 | 13 |
| | 14 | 38 | |

Melanoma imaging biomarkers exhibit strong spectral variance. Understanding the biophotonic pathologic contrast mechanisms allows targeting within the spectrum, which enables an elegant form of constrained machine learning. In building this approach, a Monte Carlo photon transport simulation was developed that exploits the optical properties of pigmented lesions for diagnosis. The observation that penetration depth is linearly related to the wavelength with a factor of 1,000 relating the two, provides a theoretical basis upon which to diagnostically target variously spaced morphologic pathologies. With the relationship that the penetration is roughly 1,000-fold longer than the wavelength of light is suggestive that the lesion in the top left has a wide area of superficial (<0.5 mm) heterogeneous pigmentation while the upper middle lesion is >1 mm-deep. The approach of correlating the spectral features with underlying morphology allows the derivation of more efficient and accurate metrics and classifiers for use with the methods of the invention.

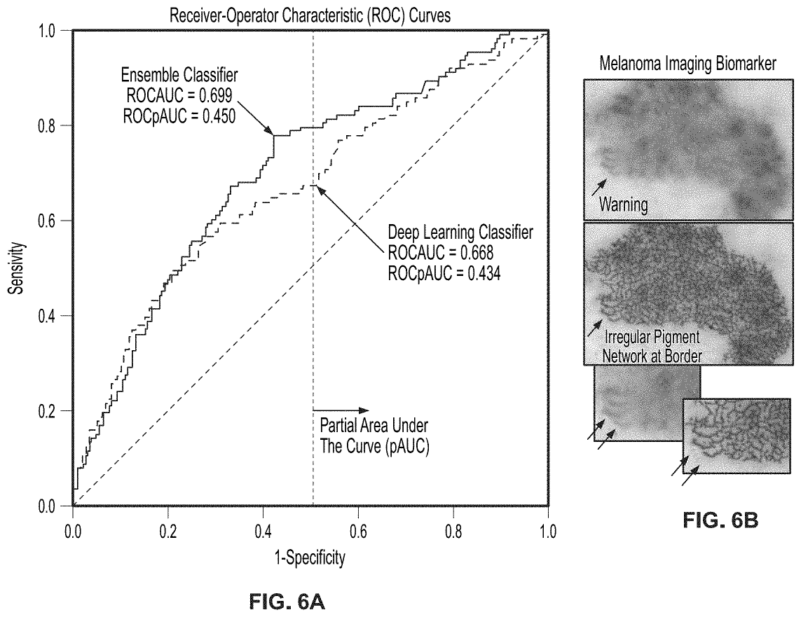

Although exemplified herein in the context of melanoma detection, digital imaging biomarkers based on visual sensory cues can be applied to any diagnostic radiology image analysis. To obtain the results summarized in FIG. 6 and Table 3, digital dermoscopy images of primary melanoma skin cancers were analyzed versus nevi that were suspicious enough to biopsy but proved histologically benign. The data set of 668 images was reduced to 349 by filtering out corrupt image data, such as images with hair or surgical ink markings overlying the lesion or lesion borders that extended beyond the image field of view, that could compromise the diagnostic.

A CNN was run versus Eclass on the same set of images (113 melanomas and 236 nevi). The CNN operated on the raw pixels in the image whereas Eclass operated on the set of imaging biomarkers, which were 30 hand-coded values automatically produced by digital image processing for each image. These 30 imaging biomarkers were designed based on real markers that dermatologists use during sensory cue integration in manual inspection of suspicious legions. Imaging biomarkers can be binary, like the presence [0 1] pixels that are blue or grey in color, integers such as the number of colors present [0-6], or continuous like the variation coefficient of branch length in a reticular pigmented network, but all imaging biomarkers used in machine learning are numbers that are high for melanoma and low for nevus.

Both CNN and Eclass learned to produce a risk score (0-1) that predicted diagnoses of melanoma (1) and nevi (0) from the noninvasive image, but Eclass uniquely implemented the language of imaging biomarkers that is designed to be visually intuitive and ultimately understandable from the doctor and patient's perspective. A graphic user interface (such as a viewfinder, for example) may be used, which is an example of visual sensory cue integration using imaging biomarker cues.

Eclass was trained and cross-validated within a Monte Carlo simulation as previously described. The convolutional neural network was based on a well-studied ResNet-50 architecture instantiated with ImageNet weights with output layers designed for binary classification. Image augmentation (flip, zoom, and rotate) and minority class (melanoma) oversampling was used during training, and test time augmentation was used during inference. The model was trained until accuracy on a validation dataset had not improved for ten epochs and the resulting model with highest validation accuracy was saved. This training procedure was repeated ten times to calculate uncertainty of ROCAUC and ROCpAUC shown in Table 3 below.

An ROC curve for deep learning classifier versus the ensemble (Eclass) classifier is depicted in FIG. 6. The images on the right hand side of FIG. 6 provide an example of the medically relevant, interpretable melanoma imaging biomarkers that may be fed to the Eclass non deep machine learning algorithms—in this case a statistical identification of abnormally long finger-like projections in the pigmented network at the peripheral border of the lesion.

Table 3 represents a statistical distribution of diagnostic performance. Eclass ran all 8 independent machine learners 1000 times in 150 seconds. CNN ran 10 times in 52 hours.

TABLE 3

| | Mean +/− SD | 95% CI |
|---|---|---|
| ROCAUC Eclass | 0.71 +/− 0.07 | [0.56 0.85] |
| ROCAUC CNN | 0.67 +/− 0.03 | [0.63 0.71] |
| ROCpAUC Eclass | 0.44 +/− 0.03 | [0.38 0.49] |
| ROCpAUC CNN | 0.44 +/− 0.01 | [0.42 0.46] |

These performance results imply that either codifying dermoscopy features into imaging biomarkers introduces information enabling Eclass to operate without access to the original pixels, or that it is not until the scale up where both classification algorithms are tested on larger data sets that the purported superiority of CNN will become evident. This is an important finding because in many cases, the size of the training set available is less than the large sets required by CNN. Thus Eclass is appropriate at least for all data sets that are underpowered for CNN and it is justified to use Eclass when at least 10 times the number (349 here) of training images (training set size) are available than the number of imaging biomarkers (30 here) developed to feed the Eclass algorithm.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

Features of the method and apparatus described herein in connection with one embodiment or one independent claim may also be combined with another embodiment or another independent claim without departing from the scope of the invention.

1. A method of dermoscopic screening of a lesion, comprising the steps of:
imaging a lesion on a subject's skin under a set of N illumination spectra to obtain a sequenced set of N images, each said image comprised of image data;
wherein the set of N illumination spectra is hyperspectral;
calculating at least one of a first type of biomarker and a second type of biomarker,
wherein the first type of biomarker comprises M imaging biomarker values and is calculated by transforming said image data of said N images into said M imaging biomarker values;
wherein the first type of imaging biomarker value varies as a function of the N illumination spectra; and
wherein the second type of biomarker is calculated from all of said N illumination spectra at each pixel, so that said second type of biomarker has only one value for said N illumination spectra at each pixel; and
applying a trained transformation algorithm to transform at least one of the first type of biomarker and the second type of biomarker into a classification indicating the likelihood that the lesion is a skin disease.

2. The method according to claim 1, wherein both the first type of biomarker and the second type of biomarker are calculated, and the trained transformation algorithm is applied to both the first and second types to obtain said classification.

3. The method according to claim 1, wherein the trained transformation algorithm comprises at least one of the following non-deep learning algorithms applied to said at least first and second type biomarkers to obtain said classification: (1) logistic regression; (2) feed-forward neural networks with a single hidden layer; (3) linear and support vector machines radial (SVM); (4) decision tree algorithm for classification problems; (5) Random Forests; (6) linear discriminant analysis (LDA); (7) K-nearest neighbors algorithm (KNN); and (8) Naive Bayes algorithm.

4. (canceled)

5. The method according to claim 1, wherein the second type of biomarker includes blood volume fraction (BVF) and oxygen saturation (O₂-Sat) to evaluate the metabolic state of tissue in the lesion.

6. The method according to claim 1, wherein a center frequency of a first spectrum of said set of N illumination spectra is separated from a center frequency of an adjacent second spectrum by approximately a half-power bandwidth of said first spectrum, such that when the N illumination spectra are normalized to have an area of unity, the first spectrum and the second spectrum intersect at respective half-power points.

7. The method according to claim 1, comprising selecting each of the N illumination spectra by dividing an entire wavelength range of said spectra into wavelength segments each approximately equal to a half-power bandwidth one of said illumination spectra, and using an illumination source emitting at a wavelength in said segment.

8. The method according to claim 1, wherein said skin disease is melanoma.

9. (canceled)

10. (canceled)

11. The method according to claim 10, comprising increasing the brightness of said LEDs at wavelengths outside the visible spectrum where an imaging sensor is less sensitive as compared to the visible spectrum.

12. A method of dermoscopic screening of lesions, comprising the steps of:
imaging a lesion on a subject's skin under a set of N illumination spectra to obtain a sequenced set of N images, each said image comprised of image data;
transforming image data of said N images into a first type of biomarker comprising M imaging biomarker values;
wherein the set of N illumination spectra is hyperspectral;
wherein each imaging biomarker varies as a function of the N illumination spectra;
applying a trained transformation algorithm to transform said M imaging biomarker values into a classification indicating the likelihood that the lesion is skin disease.

13. The method according to claim 12, wherein a center frequency of a first spectrum of said set of N illumination spectra is separated from a center frequency of an adjacent second spectrum by approximately a half-power bandwidth of said first spectrum, such that when the N illumination spectra are normalized to have an area of unity, the first spectrum and the second spectrum intersect at respective half-power points.

14. The method according to claim 12, comprising selecting each of the N illumination spectra by dividing an entire wavelength range of said spectra into wavelength segments each approximately equal to a half-power bandwidth one of said illumination spectra, and using an illumination source emitting at a wavelength in said segment.

15. (canceled)

16. The method according to claim 15, wherein said N illumination spectra range from 350 nm to 950 nm.

17. The method according to claim 16, comprising increasing the brightness of said LEDs at wavelengths outside the visible spectrum where an imaging sensor is less sensitive as compared to the visible spectrum.

18. The method according to claim 12, further comprising
calculating a second type of biomarker from all said N illumination spectra at each pixel, so that said second type of biomarker has only one value for said N illumination spectra; and
applying the trained transformation algorithm to said second type of biomarker in addition to said M imaging biomarker values to obtain said classification indicating the likelihood that the lesion is skin disease.

19. The method according to claim 18, wherein the second type of biomarker includes blood volume fraction (BVF) and oxygen saturation (O₂-Sat) to evaluate the metabolic state of tissue in the lesion.

20. The method according to claim 12, wherein the trained transformation algorithm comprises at least one of the following non-deep learning algorithms applied to said first type of biomarker to obtain said classification: (1) logistic regression; (2) feed-forward neural networks with a single hidden layer; (3) linear and support vector machines radial (SVM); (4) decision tree algorithm for classification problems; (5) Random Forests; (6) linear discriminant analysis (LDA); (7) K-nearest neighbors algorithm (KNN); and (8) Naive Bayes algorithm.

21. (canceled)

22. The method according to claim 18, wherein the trained transformation algorithm comprises at least one of the following non-deep learning algorithms applied to said first and second types of biomarker to obtain said classification: (1) logistic regression; (2) feed-forward neural networks with a single hidden layer; (3) linear and support vector machines radial (SVM); (4) decision tree algorithm for classification problems; (5) Random Forests; (6) linear discriminant analysis (LDA); (7) K-nearest neighbors algorithm (KNN); and (8) Naive Bayes algorithm.

23. The method according to claim 22, wherein the trained transformation algorithm comprises all the non-deep learning algorithms.

24.-26. (canceled)

27. An apparatus for imaging and analysis of a lesion on a subject's skin, comprising:
an illumination system controlled by a processor to sequentially illuminate a lesion on a subject's skin with N illumination spectra;
a camera controlled by a processor to obtain a sequenced set of N images of said lesion in said N illumination spectra;
a processor adapted to transform image data of said N images into a first type of biomarker comprising M imaging biomarker values;
a second processor adapted to apply a trained transformation algorithm to transform said M imaging biomarker values into a classification indicating the likelihood that the lesion is skin disease.

28. (canceled)

29. The apparatus according to claim 27, wherein a center frequency of a first spectrum of said set of N illumination spectra is separated from a center frequency of an adjacent second spectrum by approximately a half-power bandwidth of said first spectrum, such that when the N illumination spectra are normalized to have an area of unity, the first spectrum and the second spectrum intersect at respective half-power points.

30. The apparatus according to claim 27, wherein the first processor is adapted to obtain a second type of biomarker calculated from all of said N illumination spectra at each pixel, so that said second type of biomarker has only one value for said N illumination spectra at each pixel.

31. The apparatus according to claim 27, wherein more LEDs are provided in ultraviolet and infrared wavelengths where an imaging sensor is less sensitive as compared to the visible spectrum.

32. The apparatus according to claim 27, comprising a housing, wherein the housing attaches, in a self-contained unit, a transparent flat surface to position against a lesion to define a distal imaging plane, a lens, a camera, a motor, gearing; and a camera processor controlling the camera and the motor to obtain said N images.

33. The apparatus according to claim 32, wherein the housing further attaches, in the same self-contained unit, a first processor adapted to transform the N sequenced images into M biomarkers data and encrypt and transmit said M biomarkers data.

34. The apparatus according to claim 33, wherein the housing further comprises an imaging window and a space adapted to securely receive a mobile phone adapted to display an in-line view of the lesion on a display of the smart phone, and wherein the apparatus further comprises an app to connect the mobile phone to the camera processor to create a secondary display.

35.-37. (canceled)
